(19) United States
(12) Patent Application Publication
(10) Pub. No.: US 2003/0022817 A1
(43) Pub. Date: Jan. 30, 2003

(54) Additive for use in any energy supplementation, use of a protein hydrolysate to the preparation of energy supplementation and an energy supplement containing such an additive

(75) Inventors: Jorn Havskov Berthelsen, Helsingor (DK); Sven Frokjaer, Holte (DK); Bengt Saltin, Faarevejle (DK)

Correspondence Address: FINNEGAN, HENDERSON, FARABOW, GARRETT &, DUNNER LLP, 1300 I STREET, NW, WASHINGTON, DC, 20006, US

(73) Assignee: ARLA Foods AMBA

(21) Appl. No.: 10/166,036

(22) Filed: Jun. 11, 2002

Related U.S. Application Data
(63) Continuation of application No. 09/171,202, filed on Mar. 5, 1999, now abandoned.
Application No. 09/171,202 is a 371 of international application No. PCT/DK97/00177, filed on Apr. 18, 1997.

(30) Foreign Application Priority Data
Apr. 18, 1996 (DE) 0457/96

(51) Int. Cl. A61K 38/16

(52) U.S. Cl. 514/2

(57) ABSTRACT

Additive for use in any energy supplementation or metabolic nutrient in the form of a beverage or other nutrient for athletes or other persons in need of increased glycogen level, use of a protein hydrolysate to the preparation of such an energy supplementation and an energy supplement containing such an additive. The protein hydrolysate can be of animal or vegetable origin.

The present invention relates to an additive for use in any energy supplementation or metabolic nutrient, the use of a protein hydrolysate to the preparation of an energy supplementation or metabolic nutrient. The energy supplementation of the invention can be in the form of a beverage or other nutrient preparation for an athlete or another person in need of increased glycogen level and an additive for such a beverage or other nutrient for oral intake.

Athletes consume energy, primarily glycogen (carbohydrate) and later on (after 40 minutes) fat. Carbohydrate is a fast and light energy source. Consequently, carbohydrates are taken in together with liquid. In order that carbohydrates can be utilised they must pass from the blood into muscles. This transport requires insulin.

As it is, intake of water+carbohydrate increases insulin secretion moderately.

According to the invention a study has surprisingly shown that the combination water+carbohydrates+special protein hydrolysate increases insulin secretion dramatically.

This is interesting because insulin is considered an anabolic (building) hormone, and is necessary both for optimum energy uptake in muscles and brain and for uptake in (transport to) the muscles of amino acids, and is also necessary when rebuilding the spent glycogen. This means that

1) the special protein hydrolysate of the invention+carbohydrate+water increases insulin secretion and consequently the chance of supplying more energy to muscles when exercising than carbohydrate+water or carbohydrate+water+intact protein.

2) after the metabolic stress, intake of the energy supplement of the invention in the form of a beverage or other nutrient entails faster rebuilding of glycogen depots and faster rebuilding of degraded muscular proteins.

The additive according to the invention is characterized in that it is a protein hydrolysate having a degree of hydrolysis (DH) of 1-50, preferably 15-30 and especially about 25, which protein hydrolysate after oral intake rises the insulin secretion to a level above what is achieved by intake of the intact parent protein. Preferably the protein hydrolysate does not increase the gastric emptying, generally gives persons less satiety, increases the glycogen synthesis or rebuilding after depletion and reduces muscle break-down during metabolic stress, due to increased influx of amino acids into the muscle compared to what is achieved by intake of the intact parent protein.

Studies have surprisingly shown that both animal and vegetable protein is useful in the invention, which consequently comprises both types.

The protein hydrolysate can thus be of animal or vegetable origin. Useful protein hydrolysates are whey protein hydrolysates, casein protein hydrolysates, soy protein hydrolysates, wheat protein hydrolysates or pea protein hydrolysates.

The use of a protein hydrolysate to the preparation of an energy supplement or metabolic nutrient is as defined in claims 7 to 12 and the energy supplementation as defined in the claims 13 to 19.

The proteins useful in the invention can be obtained by enzymatic or non-enzymatic hydrolysis e.g. by enzymatic hydrolysis as described in the international patent application WO 92/21248.

An additive according to the invention can be used in a beverage or other nutrient product for athletes to re-establish liquid and salt balance as well as glycogen levels in the body before, under or after sport or other physical work. The beverage is preferable an isotonic fluid. The nutrient can be liquid as well as solid or be a mixture of liquids and solids, such as a paste. It can also contain sweeteners, flavours and other ingredients normally contained in beverages of this kind.

The energy supplementation of the invention contains a protein hydrolysate, a carbohydrate and optionally water or it is taken together with water.

The carbohydrate can be glucose, sucrose, maltose or maltodextrines or mixtures thereof.

The following is an example of a useful energy supplementation in the form of a beverage according to the invention.

### GERNERAL EXAMPLE

| Ingredients, pr. liter: | |
|---|---|
| Protein hydrolysate | 30 g ± 15 g |
| Carbohydrate | 70 g ± 30 g |
| Sodium | 25 mM ± 10 mM |
| Potassium | 3 mM ± 2 mM |
| Chloride | 12 mM ± 6 mM |

Carbohydrate can be glucose, sucrose, maltose, and/or maltodextrines.

Trace amounts of other mineral can be present.

### SPECIFIC EXAMPLE

| | |
|---|---|
| Lacprodan DI-3065 | 3.75% |
| SPG 30 (DE 28-32) | 3.50% |
| SPG 20 (DE 20-23) | 3.50% |
| NaCl | 0.07% |
| NaOH | 0.03% |
| Aspartam | 0.03% |
| Flavour | 0.40% |
| Malic Acid | 0.45% |
| Water | 88.27% |

### EXPERIMENTS

The following experiments have been carried out.

Rate of Gastric Emptying and Plasma Insulin Response to Protein Hydrolysate Solutions

The purpose of the present study was to determine the rate of gastric emptying of four solutions containing different quantities of proteins and protein hydrolysates (approx. 4 amino acid residuals).

Six healthy human subjects (three males and three females; 22±2.1 years; 73.2±7.8 kg weight and 177.3±10.00 cm height) participated in this study, which was approved by the local ethics committee. Test meals (600 ml) were administered through a nasogastric tube, 30 minutes after a gastric washout. Four different meals were studied in random order, over a period of 4 weeks. Solution A (control) was composed of glucose (0.025 g ml⁻¹) and NaCl (0.9%). The three other solutions contained similar quantities of protein (0.25 g (kg body mass)⁻¹) and glucose (0.025 g ml⁻¹). Solutions were composed of: B, a pea protein hydrolysate and NaCl; C, a whey protein hydrolysate and NaCl; and D, milk powder. Solutions B and C were matched for osmolality, caloric density, and nitrogen contents, but contained different amounts of essential amino acids (35.5 and 45.1%, respectively) and non-essential amino acids (64.5 and 54.9%, respectively). Each meal was delivered at 37° C., after adjusting it to a pH of approx. 7. During the 3 h that each trial lasted, blood samples were obtained every 20 min. for the assessment of the plasmatic concentrations of glucose and insulin. Gastric samples were taken every 10 min, to determine the volume remaining in the stomach using George's double-sampling technique, as applied by Beekers et al. (1988).

The rate of gastric emptying followed an exponential pattern in all cases (r=0.86-0.99). Solution A was emptied faster than the other three solutions with a half-time of 9.4±1.2 min (p<0.05). However, D emptied at a slower rate (26.4±10.0 min, p<0.05, paired t test) than both B (16.3±5.4 min) and C (17.2±6.1 min). Despite important differences in amino acid composition, both protein hydrolysate solutions emptied at a similar rate. Caloric density was closely related to the half-time of gastric emptying (r=0.96, p<0.05, n=4) and with the rate at which calories were delivered to the duodenum (r=0.99, p<0.001, n=4). The insulin response was significantly different among solutions (p<0.001, ANOVA). The plasma insulin peak elicited by the C and B (80.3±50.7 and 84.8±34.9, μmol ml⁻¹, respectively) were approx. 2- and 4-fold greater than that produced by D (47.8±13.7 μmol ml⁻¹, p<0.05, paired t test) and A (28.7±18.0 μmol ml⁻¹ p<0.05, paired t test).

The results of the present study suggest that protein hydrolysate solutions differing in the amino acid composition, but matched for temperature, volume, osmolality, acidity and caloric density, are emptied at a similar rate. In addition, the ingestion of solutions combining glucose and protein hydrolysates produce a significant and unexpected increase in plasma insulin concentration.

1. Additive for use in any energy supplementation or metabolic nutrient, characterized by the fact that it is a protein hydrolysate having a degree of hydrolysis (DH) of 1-50, which protein hydrolysate after oral intake rises the insulin secretion to a level above what is achieved by intake of the intact parent protein.

2. Additive according to claim 1, characterized by the fact that it is a protein hydrolysate which does not delay the gastric emptying, generally gives persons less satiety, optimizes the glycogen synthesis after depletion and reduces muscle break-down during metabolic stress, due to increased influx of amino acids into the muscle compared with what is achieved by intake of the intact parent protein.

3. Additive according to claim 1 or 2, characterized by the fact that it is a protein hydrolysate having a degree of hydrolysis of 15-30.

4. Additive according to claim 1 or 2, characterized by the fact that it is a protein hydrolysate having a degree of hydrolysis of about 25.

5. Additive according to any of the preceding claims, characterized in, that it is an animal or vegetable protein hydrolysate.

6. Additive according to claim 5, characterized in that it is an animal or vegetable protein hydrolysate.

7. Use of a protein hydrolysate to the preparation of a energy supplementation or metabolic nutrient, characterized in that it is a protein hydrolysate having a degree of hydrolysis (DH) of 1-50, which protein hydrolysate after oral intake rises the insulin secretion to a level above what is achieved by intake of the intact parent protein.

8. Use of a protein hydrolysate to the preparation of a energy supplementation or metabolic nutrient, characterized in that it is a protein hydrolysate which does not delay the gastric emptying, generally gives persons less satiety, optimizes the glycogen synthesis after depletion and reduces muscle break-down during metabolic stress, due to increased influx of amino acids into the muscle compared with what is achieved by intake of the intact parent protein.

9. Use of a protein hydrolysate to the preparation of a energy supplementation or metabolic nutrient, characterized by the fact that it is a protein hydrolysate having a degree of hydrolysis of 15-30.

10. Use of a protein hydrolysate to the preparation of a energy supplementation or metabolic nutrient, characterized by the fact that it is a protein hydrolysate having a degree of hydrolysis of about 25.

11. Use of a protein hydrolysate to the preparation of a energy supplementation or metabolic nutrient, characterized in, that it is an animal or vegetable protein hydrolysate.

12. Use of a protein hydrolysate to the preparation of a energy supplementation or metabolic nutrient, characterized in that it is a whey protein hydrolysate, a casein protein hydrolysate, a soy protein hydrolysate, a wheat protein hydrolysate or a pea protein hydrolysate.

13. Energy supplementation in the form of a beverage or other nutrient for human beings characterized in, that it contains an additive according to any of the claims 1-6 and a carbohydrate.

14. Energy supplementation according to claim 13, characterized in that the additive is a protein hydrolysate having a degree of hydrolysis (DH) of 1-50, which protein hydrolysate after oral intake rises the insulin secretion to a level above what is achieved by intake of the intact parent protein.

15. Energy supplementation according to claim 13, characterized in that the additive is a protein hydrolysate which does not delay the gastric emptying, generally gives persons less satiety, optimizes the glycogen synthesis after depletion and reduces muscle break-down during metabolic stress, due to increased influx of amino acids into the muscle compared with what is achieved by intake of the intact parent protein.

16. Energy supplementation according to claim 13, characterized in that the additive is a protein hydrolysate having a degree of hydrolysis of 15-30.

17. Energy supplementation according to claim 13, characterized in that that the additive is a protein hydrolysate having a degree of hydrolysis about 25.

18. Energy supplementation according to claim 13, characterized in that the additive is an animal or vegetable protein hydrolysate.

19. Energy supplementation according to claim 13, characterized in that the additive is a whey protein hydrolysate, a casein protein hydrolysate, a soy protein hydrolysate, a wheat protein hydrolysate or a pea protein hydrolysate.

20. Energy supplementation according to any of the claims 13 to 19, characterized in that the carbohydrate is glucose, sucrose, maltose or a maltodextrine or mixtures thereof.
